SEQUENCE LISTING (6 sequences)

SEQ ID NO: 1 - DNA, 6654 bp, Artificial Sequence
  SARS-COV-2-N
  CDS (960)..(2216)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccagctagcgtttaaacttaagcttggtaccgccatggactacaaggacgacgacgacaaggggcggccgccctctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - protein, 418 aa, Artificial Sequence
  Synthetic Construct
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - DNA, 9218 bp, Artificial Sequence
  SARS-COV2-S
  CDS (960)..(4778)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccagctagcgtttaaacttaagcttggtaccgccatggactacaaggacgacgacgacaaggggcggccgccctttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataagctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - protein, 1272 aa, Artificial Sequence
  Synthetic Construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 6063 bp, Artificial Sequence
  SARS-COV-2-M
  CDS (960)..(1625)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccagctagcgtttaaacttaagcttggtaccgccatggactacaaggacgacgacgacaaggggcggccgcaagcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - protein, 221 aa, Artificial Sequence
  Synthetic Construct
ADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

(19) United States
(12) Patent Application Publication
(10) Pub. No.: US 2023/0227844 A1
(43) Pub. Date: Jul. 20, 2023

(54) CELL-MEDIATED SARS-COV-2 VACCINES, AND PREPARATION AND USE THEREOF

(71) Applicant: Xiushan YIN, Shenyang (CN)

(72) Inventors: Xiushan YIN, Shenyang (CN); Junhua LIU, Shenyang (CN)

(21) Appl. No.: 18/010,407

(22) PCT Filed: Jun. 15, 2020
(86) PCT No.: PCT/CN2020/096030

§ 371 Date: Dec. 14, 2022

(51) Int. Cl. C12N 15/85; C12N 5/10; C12N 15/88; A61P 31/14; C07K 14/165

(52) U.S. Cl. CPC C12N 15/85; C12N 5/10; C12N 15/88; A61P 31/14; C07K 14/165; A61K 2039/5156

(57) ABSTRACT

Provided are a cell-mediated SARS-COV-2 vaccine and a preparation method therefor, the steps therefor including: the construction of a SARS-COV-2 specific antigen vector presented by stem cells, and the modification and assembly with the stem cells. Two weeks after mouse immunization, approximately 50% of the mice have in vivo antibodies that show a strong positive expression, and the most significant of which being an N-gene modified stem cell vaccine.

### TECHNICAL FIELD

The present disclosure relates to the technical field of cellular and genetic engineering, and especially includes construction of a SARS-COV-2-specific vector presented by cells, and a SARS-COV-2 vaccine mediated by improved stem cells.

### BACKGROUND

Coronavirus disease 2019 (COVID-19) has now spread all over the world, resulting in the infection of nearly millions of people worldwide. It has now been declared a “pandemic” by the World Health Organization. The main pathogen causing this major epidemic is a novel severe acute respiratory syndrome-related coronavirus-2, which is referred to as “SARS-COV-2”. The typical symptoms of infected people include headache, fever, cough, body aches and fatigue, etc. Mild patients generally have unpleasant symptoms similar to influenza virus infection, while severe patients can have local pulmonary dysfunction, respiratory failure, or the like. By sequencing the nucleic acid of the virus isolated from patients suffering from COVID-19, it has been found that the virus is an enveloped positive-sense single-stranded RNA (ssRNA+) virus having a high sequence homology to the sequences of the virus that caused severe acute respiratory syndrome (SARS) in 2003, of the virus that caused Middle East respiratory syndrome-(MERS) in 2012, and of viruses found in bat, all of which belong to beta coronaviruses. The nucleic acid and protein structural characteristics of the virus are continuously revealed that the virus contains four major structural proteins, namely, spike (S) protein, membrane (M) protein, nucleocapsid (N) protein and envelope (E) protein, as well as multiple nonstructural proteins, such as ORF1a/b, ORF3a, etc. During infection and immunization, the virus utilizes its spike protein to bind tightly to the angiotensin-converting enzyme 2 (ACE2) protein on the surface of human host cells. Upon the viral RNA being released into the cells, nucleoplasmic components in the host cells assist in retranslation, packaging and release of the viral particle, which in turn adapts to and infects human cells. However, there are still no clear research results on the most original traceability of the virus, cross-species transmission, and intermediate hosts.

In the face of the sudden epidemic, besides diagnostic work such as early nucleic acid detection (RT-PCR, RT-LAMP, etc.), antibody detection (such as colloidal-gold method, enzyme-linked immunosorbent assay), imaging monitoring and new generation detection methods (such as CRISPR-SHERLOCK technology by Zhang Feng's team), the research and development of vaccines related to SARS-COV-2 is also the critical work for epidemic prevention and control. So far, there is no medication specific to the virus in the world, and the research of related vaccines is also a thorny problem in medicine.

At present, there are many directions for the vaccine research and development of SARS-COV-2 in the world, including inactivated virus vaccines, nucleic acid vaccines, viral vector vaccines, protein vaccines, etc. In China, the currently reported adenovirus vector vaccine has entered phase II clinical trial, as well as the inactivated vaccine currently developed by Sinopharm Group. Both vaccines are obtained by traditional vaccine research and development approaches. The inactivated virus vaccine is an inactivated virus that is to be injected to stimulate the body to produce an immune response. This kind of inactivated vaccine is easy to produce, but requires high dose of multiple immunization injections, and has a short immunization period and a single immunization route. Secondly, the adenovirus vector vaccine is essentially a modified adenovirus as an expression vector into which partial sequence of the structural protein of SARS-COV-2, mainly S protein, is inserted using genetic engineering to produce the essential protein of SARS-COV-2 in vivo, which then stimulates the immune system to resist the virus. Meanwhile, adenoviral vectors may develop immune resistance in some people, thereby limiting or impairing the effectiveness of the vaccine of interest. Herein, based on the above viewpoints and the superiority of stem cells, especially MSCs, in the treatment of diseases, the present disclosure provides a new idea for developing a SARS-COV-2 vaccine, that is to use stem cells as the main mediating carrier to induce the immune system in vivo to produce corresponding antibodies and establish long-term immune memory.

### Technical Problem

At present, the vaccine research and development platforms related to COVID-19 mainly involve traditional inactivated or attenuated vaccines, nucleic acid vaccines, viral vector vaccines, protein vaccines, etc. Based on this, research is moving on by many research and development institutions. Some of the SARS-COV-2 vaccines may be used to repeatedly immunize the body, and some may even cause discomfort to the body. Yet, there has been no relevant report on the research and development route of a SARS-COV-2 vaccine mainly based on a cell delivery platform.

### Technical Solutions

The present disclosure provides a novel cell-based research and development route for a SARS-COV-2 vaccine. It mainly includes: 1) constructing a SARS-COV-2-specific S vector, M vector and N vector presented by the cells; 2) preparing modified cells; and 3) expressing and secreting soluble proteins to generate immune memory in an experimental animal.

### Beneficial Effect

“A cell-mediated SARS-COV-2 vaccine” refers to a SARS-COV-2 vaccine that is developed using cells as a new delivery platform, so that stem cells carrying an essential viral protein can enter the body to stimulate the body to produce effective immune resistance. In theory, the stem cell vaccine is developed using a similar principle to that for the nucleic acid vaccine, except that stem cell vaccines do not directly produce some viral antigenic proteins using animal or human cells. In a word, it is a relatively safe novel vaccine technology.

The adenovirus vector COVID-19 vaccine and the inactivated virus COVID-19 vaccine developed by Sinopharm Group that have entered the phase II clinical trial are both vaccines in the traditional sense. In theory, the inactivated virus vaccine is a virus that is chemically inactivated or heat-inactivated to stimulate the body to produce an immune response. This vaccine is relatively easy to produce, but requires high dose and has a short immunization period, as well as the virus expansion, which is likely to cause a greater risk of recontamination. In theory, for the adenovirus vector vaccine, a modified adenovirus is used as a mediating carrier, into which nucleic acid sequence encoding the essential protein of SARS-COV-2, mainly S protein currently, is inserted using genetic engineering, and which is injected into the body and then stimulates the immune system to resist the virus. Essentially, the treated virus is used as a mediating carrier for both vaccines. Meanwhile, adenoviral vectors may develop immune resistance in some people, thereby limiting or impairing the effectiveness of the vaccine of interest.

In comparison with the prior art, the present disclosure utilizes stem cells to present and secrete some of the essential antigenic proteins of SARS-COV-2. First, the present disclosure can accelerate the production of specific antibodies and effectively avoid adverse immune responses caused by viral vector vaccine injections. Meanwhile, the present disclosure enables the delivery of antigen proteins, such as S protein, M protein or N protein, etc., to multiple targets, so as to generate broad-spectrum pleiotropic antibodies.

Second, in view of the good clinical significance of stem cell therapy, especially MSCs, and the continuous elimination of the injected cells by the body's immune system over time, the SARS-COV-2 vaccine mediated by stem cells is relatively safe in the human body.

Third, studies have shown that most (about 50% or more) of the surface proteins of SARS-COV-2 virus are glycosylated, generally resulting in a greatly reduced success rate of one-time immunization with the traditional vaccines. The SARS-COV-2 vaccine using stem cells as a mediating carrier will be subjected to the protein glycosylation in the stem cell expression system, which can well simulate the process of real virus expression in host in vivo and will greatly improve success rate of one-time immunization as compared with the traditional vaccines.

Fourth, compared with other types of vaccines, the cell-mediated SARS-COV-2 vaccine has a huge advantage in timeliness, with a great number of antibodies detectable in experimental animals within two weeks.

### BRIEF DESCRIPTION OF THE DRAWINGS

FIG. 1 is a diagram showing the preparation process of a cell-mediated SARS-COV-2 vaccine and related analysis route, including the preparation of cell-mediated modified vector, the delivery process of related cells, the immune injection and the detection and analysis of related immune antibodies, etc.

FIG. 2 is a diagram showing the results of construction of the SARS-COV specific S, M and N vectors presented by stem cells. FIG. 2A shows the insertion sites and related images of the SARS-COV-2-specific S, M, and N sequences presented by stem cells; FIGS. 2B and 2C show fragments obtained by specific amplification of S and M, and N genes (carrying NotI and XbaI specific restriction sites) respectively; FIG. 2D shows fragment obtained by digestion of pc3.1 scaffold vector with the same enzymes, i.e. NotI and XbaI; FIG. 2E shows an amplified fragment of the M gene which is inserted by homologous recombination (carrying specific restriction sites NotI and XbaI).

FIG. 3 is a diagram showing the optimum transfection and preparation method and the detection results of the antibody in the serum immunized by stem cell N-type vaccine; FIGS. 3A and 3B are the comparisons of transfection effects using different ratios of plasmids to transfection reagents. FIG. 3A shows the density of fluorescence-carrying cells measured by flow cytometry, wherein the cells transfected with various ratios of plasmids to Lipofectamine® 2000 reagent of 3 μg:6 μL; 3 μg:9 μL; 3 μg:12 μL; and 3 μg:15 μL shows transfection effects of 40.77%, 50.96%, 61.49% and 62.30%, respectively. The most efficient transfection ratio is 3 μg:15 μL.

FIGS. 3C and 3D show detection of the antibody in sera of immunized mice after the first and second injections. The first batch involves N-SI 33 (N gene-subcutaneously injected male mice No. 33), N-MI 35 (N gene-intramuscularly injected male mice No. 35), N-SI 41 (N gene-subcutaneously injected female mouse No. 41), and N-MI 43 (N gene-intramuscularly injected female mouse No. 43). The second batch involves N-SI 30 (N gene-subcutaneously injected male mice No. 30), N-MI 28 (N gene-intramuscularly injected male mice No. 28), N-SI 5 & 7 (N Gene-subcutaneously injected, female mice Nos. 5 and 7), and N-MI 4&12 (N gene-intramuscularly injected female mice Nos.4 and 12). The experimental results showed that the cell-mediated SARS-COV-2 vaccine can induce the production of antibodies in mice, and the mice injected intramuscularly had the highest positive rate of antibodies in testing the serum of immunized mice.

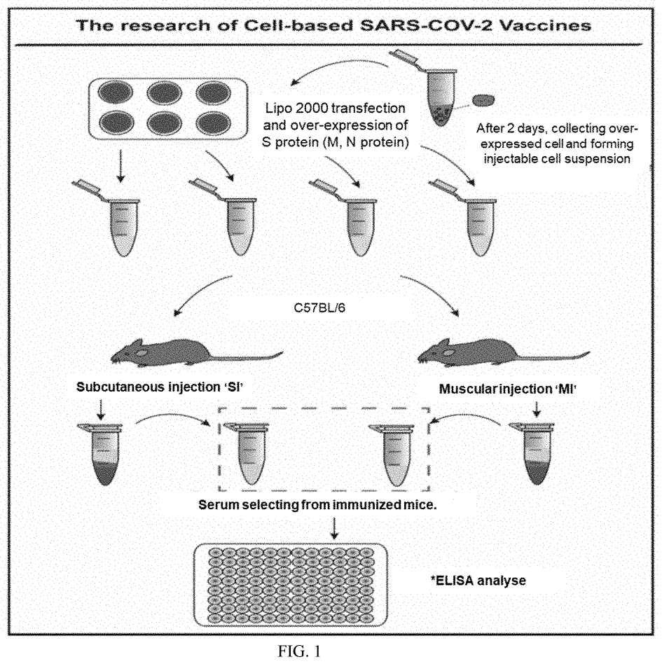

### DETAILED DESCRIPTION OF THE EMBODIMENTS

In this specification, the accurate construction and identification of SARS-CoV-2-specific S, M and N vectors presented by cells are shown in the optimal steps, and the cell transfection conditions related to the preparation process of the cell-mediated COVID-19 vaccine and injection dosage of the cell-mediated COVID-19 vaccine are all shown in optimal modes. For specific implementation steps, refer to the embodiments of the present disclosure.

### EXAMPLES OF THE PRESENT DISCLOSURE

1. The present disclosure will be further elaborated below in conjunction with the specific embodiments. Specifically, some of the essential proteins of SARS-CoV-2 as antigenic epitopes are constructed in the pc3.1 eukaryotic expression vector, transferred into a specific cell system, and then initially forms a stem cells-mediated vaccine, which is injected into mice to test immune responses and antibody production.

2. The experimental reagents and consumables used herein include as follows: Q5® High-Fidelity DNA Polymerase (NEB #M0491), NotI-HF (NEB #R3189S), XbaI (NEB #R0145V), Quick ligase (NEB #M2200S), Trelief™ 5a Chemically Competent Cell (TSINGKE TSC01), Endo-Free Plasmid Mini Kit II (OMEGA D6950-02), Lipofectamine 2000 (Invitrogen 11668-019), EasyGeno Assembly Cloning kit (Tiangen Biotech (Beijing) Co., Ltd VI201), DMEM High Glucose (Gibco C11995500BT), Opti-MEM I Reduced Serum Medium no phenol red (Gibco 11058021), PBS (Tianjin Haoyang Biological Manufacture Co., Ltd, TBD PB2004Y), Penicillin/Streptomycin solution 100× (Meilunbio MA0110), Fetal Bovine Serum (Gibco), six-well plate and 10 cm petri dish (Thermo Fisher SCIENTIFIC), 100 um cell strainer (Corning FALCON 352360), 1 ml disposable medical syringe (Jiangxi Hongda Medical Equipment Group Ltd.), and ELISA reagents (from Immunology Research Group of Jilin University).

3. Cell Lines and Mouse Strains

293T cell line was cultured under the following specific culture conditions. The cell line was cultured in DMEM High Glucose containing 10% FBS. All culture plates (or dishes) containing cells according to the present disclosure were cultured in a normoxic incubator in 5% CO₂ at 37° C.

C57BL/6 strain mice (6-8 weeks old, provided by Liaoning Changsheng Biotechnology Co., Ltd.) were selected as experimental mice.

4. Example 1. Construction and Identification of SARS-CoV-2-Specific S and N Vectors Presented by Cells.

Referring to the genome sequence of SARS-CoV-2 virus annotated on NCBI, nucleic acid sequences of S, M and N proteins were selected for gene synthesis by Suzhou GENEWIZ Inc. The synthetic gene was inserted into a pUC57 vector. First, genes of interest were amplified by PCR (see Table 2 for specific amplification primer sequences), and the bands in size of interest were recovered and purified. A pc3.1-Falg-XXX vector and the purified S and N fragments were digested overnight with restriction endonucleases NotI-HF and XbaI in a water bath at 37° C. The next day, the vector of interest and the gene fragment containing the restriction site were recovered from the gel and ligated. The resultant plasmid was transformed into Trelief™ 5a competent cells. Single colonies were picked and sequenced to verify the successful construction. Finally, the vectors named pc3.1-Flag-SARS_COV_2-S and pc3.1-Flag-SARS_COV_2-N were successfully constructed by following specific steps.

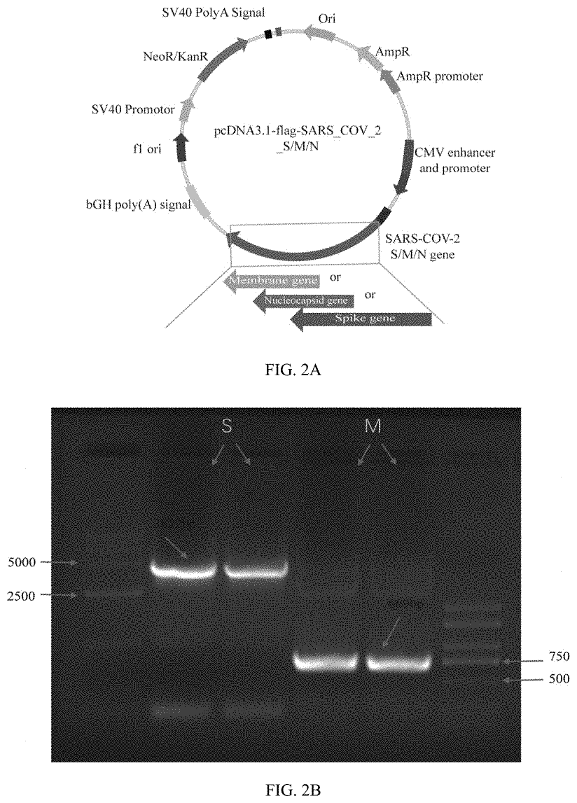

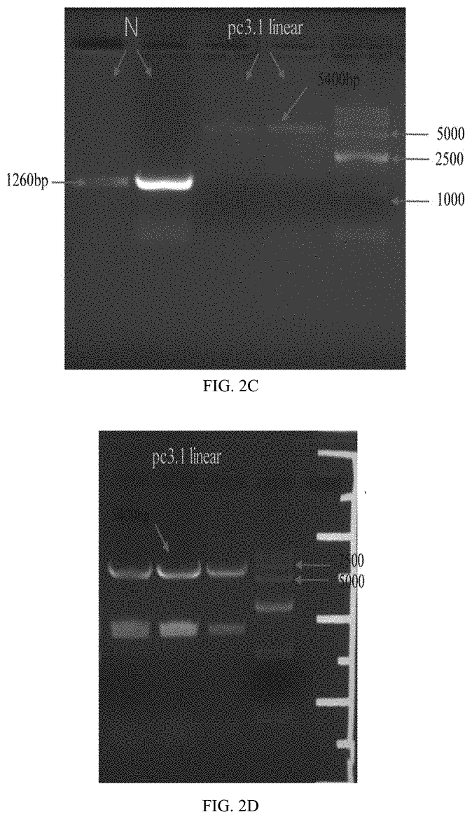

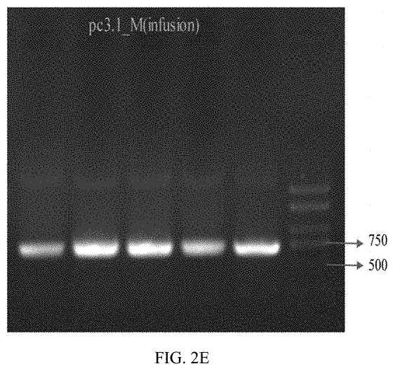

(1) Amplification of fragments S and N of interest: puC57-S was used as the amplification template. Q5 high-fidelity enzyme amplification system, namely 5× Q5 Reaction Buffer 10 μL, 10 mM dNTPs 1 μL, template puC57-S 2 μL, pcDNA3.1-Flag-S F and R 2.5 μL each, and Q5 enzyme, was added. Distilled water was added to make up to 50 PCR protocol: pre-denaturation at 98° C. for 30 s; then 35 cycles of 98° C. for 10 s, 56° C. for 30 s, and 72° C. for 2 min; then final extension at 72° C. for 5 min; and then holding at 4° C. Similarly, using puC57-S as the amplification template, N fragment was amplified according to the components and protocol in Table 3. The amplified products S and N were recovered in bands of 3,822 bp and 1,260 bp respectively by agarose gel electrophoresis. The results are shown in FIGS. 2A, 2B and 2C.

TABLE 1
Components and corresponding PCR protocol for N amplification.
Components, amount and corresponding PCR protocol for N amplification

| | | |
|---|---|---|
| 5X Q5 Reaction Buffer | 10 μL | 98° C. for 30 s; |
| 10 mM dNTPs | l μL | 98° C. for 10 s, |
| 10 μM pcDNA3.1-Flag-S F Primer | 2.5 μL | 60° C. for 30 s, |
| 10 μM pcDNA3.1-Flag-S R Primer | 2.5 μL | 72° C. for 2 min; |
| | | Goto2: x35 cycles; |
| puC57-N template | 0.5 μL | 72° C. for 2 min; |
| Q5 High-Fidelity DNA Polymerase | 0.5 μL | holding at 4° C. |
| 5X Q5 High GC Enhancer | 10 μL | |
| Nuclease-Free Water | to 50 μL | |

(2) Enzyme digestion of the product of interest and the backbone vector pc3.1-flag-xxx into which the product of interest is to be inserted: to each of recovered S and N fragments, 0.2 μL of NotI-HF and XbaI and 2 μL of corresponding 10× Cutsmart Buffer solution were added to make up complete 20 μL digestion systems. The systems were incubated overnight at 37° C. The pc3.1 vector as the backbone was also digested at the same sites. The larger fragment in band of 5400 bp was recovered by electrophoresis (FIG. 2D).

(3) After the digestion was completed, the three products were recovered using DNA gel recovery kit from Omega and measured for concentration using Narodrop2000.

(4) ligation and transformation: to 6 μL of S fragment and 3 μL of linearized backbone vector pc3.1-flag-xxx, 1 μL of quick ligase and 10 ul of corresponding 2× Ligation buffer were added and subject to ligation at room temperature for 15 min. 50 μL of Trelief™ 5a competent cells were thawed on ice. 5 μL of ligation product was added to the cells and left standing for 30 min. Then the cells were heat shocked at 42° C. for 90 s, and incubated on ice again for 5 min. Finally, 600 μL of non-resistant LB medium was added to the cells before incubating the cells in a shaker at 250-300 rpm for 60 min.

(5) Screening and obtaining single positive colonies: 200 μL of the above-mentioned non-resistant culture was spread on agar plates with ampicillin and incubated at 37° C. overnight. Then, single positive colonies could be screened and randomly picked. 6 EP tubes containing the medium with ampicillin were prepared. The colonies were picked gently with a 10 μL pipette tip into the tubes and incubated by quick shaking for 5 hours. The bacteria culture was verified by PCR. The verified bacteria could be subjected to plasmid miniprep extraction. The extracted plasmids were sequenced for further verification. The plasmids verified by sequencing were subjected to transfection-grade plasmid midi prep extraction and purification.

TABLE 2
Primer sequences involved in the
construction of vectors for cell vaccine series.

| Name of | Sequence | |
|---|---|---|
| primer | of primer (5′-3′) | Remark |
| pcDNA3.1- | 5′-GGGCGGCCGCCCTT | 3842 bp |
| Flag-S F | TGTTTTTCTTGTTTTAT | for amplifying |
| | TGC-3′ | S sequences, |
| pcDNA3.1- | 5′-CCCTCTAGAGCTTA | containing |
| Flag-S R | TGTGTAATGTAATTTGA | NotI and XbaI |
| | CTC-3′ | restriction |
| | | sites, |
| | | respectively |
| pcDNA3.1- | 5′-GGGCGGCCGCCCTC | 1278 bp |
| Flag-N F | TGATAATGGACCCCA-3′ | for amplifying |
| pcDNA3.1- | 5′-CCCTCTAGATTAGG | N sequences, |
| Flag-N R | CCTGAGTTGAGTC-3′ | containing |
| | | NotI and XbaI |
| | | restriction |
| | | sites, |
| | | respectively |
| pcDNA3.1- | 5′-GACGACAAGGGGCG | 666 bp |
| fusion-M F | GCCGCAAGCAGATTCCA | for amplifying |
| | ACGGTACTAT-3′ | N sequence, |
| pcDNA3.1- | 5′-TTTAAACGGGCCCT | being inserted |
| fusion-M R | CTAGATTACTGTACAAG | by |
| | CAAAGCAAT-3′ | homologous |
| | | recombination |

### Example 2 Construction and Identification of SARS-CoV-2-Specific M Vectors Presented by Cells

Since the M gene contains both NotI and XbaI restriction sites at the end of the vector, another way for inserting the M gene sequence was utilized for homologous recombination. First, the synthetic M gene sequence (see attachment 2) was used as the template. Q5 high-fidelity enzyme amplification system, i.e. 5× Q5 Reaction Buffer 10 μL 10 mM dNTPs 1 μL, template puC57-M 2 μL, pcDNA3.1-Flag-M F and R 2.5 μL each, and Q5 enzyme, was used for amplification and distilled water was added to make up to 50 μL. PCR protocol: pre-denaturation at 98° C. for 30 s; then 35 cycles of 98° C. for 10 s, 60° C. for 30 s, and 72° C. for 2 min; then final extension at 72° C. for 5 min; and then holding at 4° C. The product was subjected to electrophoresis. The fragment of interest recovered from gel is 666 bp in size (as shown in FIG. 2E). Without enzyme digestion, the M-specific amplification product was directly ligated with the linearized backbone vector pc3.1-flag-xxx mentioned in Example 1 using EasyGeno Assembly Cloning kit. The subsequent routine transformation and screening of positive bacteria were carried out according to those described in Example 1. The clone strain with M gene was successfully obtained, and the vector it carried was named pcDNA3.1-flag-SARS_COV_2_M (INfusion).

### Example 3 Optimization Experiments for Cell Line Transfection Efficiency

Pre-treatment of cells for transfection: Cells were passaged into six-well plates. The cell density in each well should be consistent. It should be ensured that the cell density before transfection is about 80-90%.

Transfection operation: (1) 14 μg of plasmid was dissolved in 700 μL of serum-free medium in a fresh EP tube (concentration c=20 ng/ul).

(2) 4 fresh EP tubes with four concentrations (6, 9, 12, and 15 μL) recommended for the Lipofectamine 2000 kit respectively were used to determine the new optimal ratio of the cell line. 6, 9, 12, and 15 μL of Lipofectamine® Reagent were diluted in 150 μL of serum-free medium (DMEM® Medium) in the EP tubes, respectively.

(3) During transfection, the diluted plasmid solution was gently pipetted into the EP tube containing Lipofectamine 2000 and mixed by shaking the tube gently, that is, a system of 150 μL (including 3 μg)+150 μL was incubated at room temperature for 5 min to 10 min.

(4) The a six-well plate to be transfected was refreshed with 1.5 ml of fresh medium. Then 300 μL of the incubated plasmid-lipo2000 mixture was added to one each well of the six-well plate (i.e., 3.0 μg per well). The plate was shaken well, and then moved back into the 37° C. incubator for incubation.

(5) Transfection efficiency test by flow cytometry 48 hours after transfection.

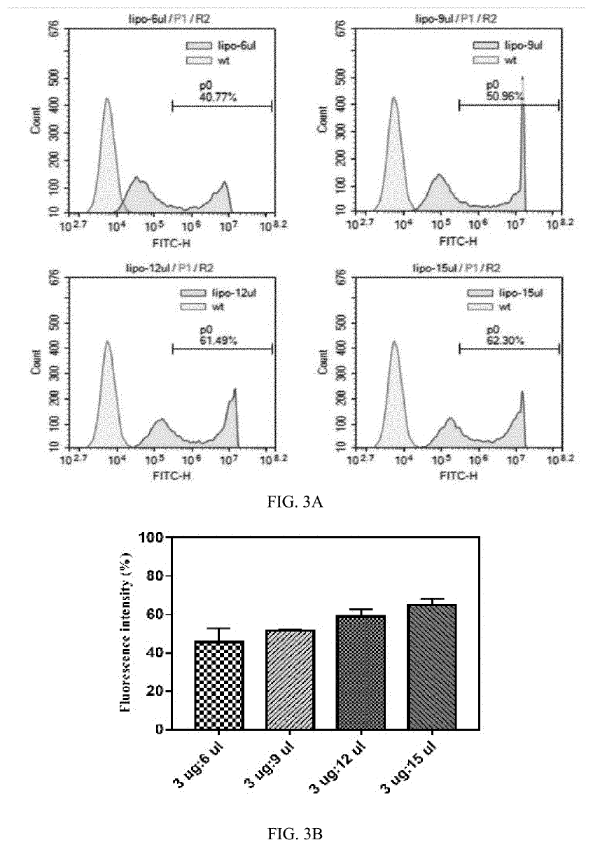

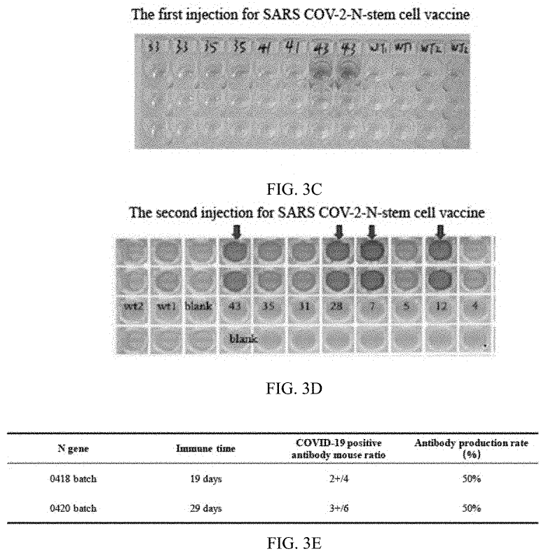

The experimental results of determining the optimal transfection ratio are shown in FIG. 3A. The results show that for human embryonic kidney cells (293T cells), when the ratio of plasmid to Lipofectamine 2000 is 3 μg:15 μL, the transfection efficiency is the best with the efficiency index as high as 60-70%. Therefore, subsequent transfection was performed based on the optimal ratio.

### Example 4 Preparation of Cell-Mediated Sars-Cov-2 Vaccine

150 ul serum-free medium was added into 4 EP tubes each. 3 μg pcDNA3.1-flag-2019_nCOV_S, pcDNA3.1-flag-2019_nCOV_N, pcDNA3.1-flag-SARS_COV_2_M (INfusion), and a mixture thereof (in a ratio of 1:1:1) were added to the tubes respectively with well mixing.

15 μL Lipofectamine 2000 reagent was added into each of 4 new EP tubes with the same labels as the 4 tubes as mentioned above. After gently and well mixing, the plasmid-containing solution was transferred into the corresponding EP tubes containing Lipofectamine 2000, and left standing for 10 min.

The six-well plate to be transfected was refreshed with 1.5 ml of fresh medium. Then 300 μL of the incubated plasmid-lipo2000 mixture was added dropwise dispersedly to one well of the six-well plate. The plate was shaken well, and then moved back into the 37° C. incubator for incubation. Cells were harvested after 48 hrs.

### Example 5 Procedures for Immunizing Experimental Animals

Herein, black C57BL/6 mice were used in the animal injection experiment. Mice of different genders were randomly divided into two groups according to the injection mode (intravenous and subcutaneous). Each of the group was subdivided into 2-3 parallel groups. Each mouse was named according to the gene-injection mode-gender, and was also randomly labeled on ears. The specific operation steps are as follows.

(1) The cells transfected with the eukaryotic expression vectors pc3.1-Flag-S, M and N of the cell-mediated SARS-COV-2 vaccine was digested with 0.25% Trypsin-EDTA and centrifuged. The cells in the bottom were collected and resuspended with an appropriate amount of PBS solution. After counting using a hemocytometer, a cell suspension of 200,000 cells/μL was prepared and left standing on ice.

(2) C57BL/6 mice aged 6-8 weeks were injected intramuscularly or subcutaneously, respectively. The mice were disinfected with 75% alcohol and injected with 50 μL (containing 1 million stem cells) of the modified stem cell suspension using a 1 ml syringe at right rear thigh, or was injected with the same amount of modified stem cell suspension as above at the flank or head and neck for subcutaneous injection. Mode of registering: gender-gene name-injection mode-labelling number

### Example 6 Detection of Antibodies in Sera from Immunized Animals Following Cell-Mediated Vaccine Injection

The immune serum antibodies were detected by ELISA. The pre-coated plates were provided by Immunology Research Group of Jilin University. The specific operation process is as follows.

(1) Sample loading: a pre-coated plate was fixed on a plate rack. 2-3 wells were set as negative control and 2 wells as blank control. For the experimental sample group, 100 μL of sample diluent (except blank) was first added to each well, followed by 5 μL of the sample serum to be tested. The plate was mixed well by shaking and incubated for about 60 min in a 37° C. incubator.

(2) Washing: the liquid in the wells was discarded. 300 μL of washing solution was added for washing 5 times. Specifically, the plate was inverted to throw the liquid in the wells into trash can in one-off form. Then washing solution, which was prepared in advance and placed in a box similar to a PCR tube rack, was added using a 100 μL multichannel pipette. The coated plate was washed for 30 to 60 s by gently shaking.

(3) Secondary antibody addition: 100 μL (1:10000 dilution) of secondary antibody (goat anti-mouse monoclonal antibody containing horseradish peroxide) was added to each well. The plate was incubated in a 37° C. incubator for about 30 min.

(4) Washing: the liquid in the wells was discarded. 300 μL of the washing solution was added for washing 5 times.

(5) Color development: 50 μL of substrate buffer was added to each well, followed by 50 μL of substrate solution, for well mixing. The plate was incubated at 37° C. for 15 min.

(6) Stop: 50 ul of stop solution was added to each well, and after well mixing for 15 min, OD value at a wavelength of 450 nm was measured by a microplate reader. or alternatively, color contrast image scanning was performed.

The sera of the immunized mice were analyzed by ELISA. The results are shown in FIGS. 3C and 3D: After testing the mouse serum two weeks after immunization and the mouse serum four weeks after immunization, antibodies can be detected effectively in almost all immunized mice, among which about 50% of the mice showed strong positive expression of antibodies, especially that immunized with the cell vaccines modified with N gene. Since the data statistics table in FIG. 3E is an illustration of the results after one injection, the cell-based SARS-COV-2 vaccine prepared according to the present disclosure has better immune efficacy.

In addition, only the preferred implementation of the present disclosure is described above. For skilled one in the art, any improvement or supplement based on the conceptual framework described in this experiment, including cell vaccines, not limited to stem cells, against other viruses, bacteria and pathogens should also be regarded as within the protection scope of the present disclosure.

Content in Sequence Listing

Complete maps of SARS-CoV-2-specific S-, M-, and N-vectors presented by cells.

>pc3.1-Flag-SARS_COV_2-N.
gacggatcgggagatctcccgatcccctatggtgcactctcagtacaatctgctctgatgccgcatagttaagcc
agtatctgctccctgcttgtgtgttggaggtcgctgagtagtgcgcgagcaaaatttaagctacaacaaggcaaggcttgaccga
caattgcatgaagaatctgcttagggttaggcgttttgcgctgcttcgcgatgtacgggccagatatacgcgttgacattgattattg
actagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccat
tgacgtcaatgggtggagtatttacggtaaactgcccacttggcagtacatcaagtgtatcatatgccaagtacgccccctattga
cgtcaatgacggtaaatggcccgcctggcattatgcccagtacatgaccttatgggactttcctacttggcagtacatctacgtatt
agtcatcgctattaccatggtgatgcggttttggcagtacatcaatgggcgtggatagcggtttgactcacggggatttccaagtct
ccaccccattgacgtcaatgggagtttgttttggcaccaaaatcaacgggactttccaaaatgtcgtaacaactccgccccattga
cgcaaatgggcggtaggcgtgtacggtgggaggtctatataagcagagctctctggctaactagagaacccactgcttactggc
ttatcgaaattaatacgactcactatagggagacccagctagcgtttaaacttaagcttggtaccgccatggactacaaggacgac
gacgacaaggggcggccgccctctgataatggaccccaaaatcagcgaaatgcaccccgcattacgtttggtggaccctcag
attcaactggcagtaaccagaatggagaacgcagtggggcgcgatcaaaacaacgtcggccccaaggtttacccaataatact
gcgtcttggttcaccgctctcactcaacatggcaaggaagaccttaaattccctcgaggacaaggcgttccaattaacaccaata
gcagtccagatgaccaaattggctactaccgaagagctaccagacgaattcgtggtggtgacggtaaaatgaaagatctcagtc
caagatggtatttctactacctaggaactgggccagaagctggacttccctatggtgctaacaaagacggcatcatatgggttgc
aactgagggagccttgaatacaccaaaagatcacattggcacccgcaatcctgctaacaatgctgcaatcgtgctacaacttcct
caaggaacaacattgccaaaaggcttctacgcagaagggagcagaggcggcagtcaagcctcttctcgttcctcatcacgtagt
cgcaacagttcaagaaattcaactccaggcagcagtaggggaacttctcctgctagaatggctggcaatggcggtgatgctgct
cttgctttgctgctgcttgacagattgaaccagcttgagagcaaaatgtctggtaaaggccaacaacaacaaggccaaactgtca
ctaagaaatctgctgctgaggcttctaagaagcctcggcaaaaacgtactgccactaaagcatacaatgtaacacaagctttcgg
cagacgtggtccagaacaaacccaaggaaattttggggaccaggaactaatcagacaaggaactgattacaaacattggccgc
aaattgcacaatttgcccccagcgcttcagcgttcttcggaatgtcgcgcattggcatggaagtcacaccttcgggaacgtggttg
acctacacaggtgccatcaaattggatgacaaagatccaaatttcaaagatcaagtcattttgctgaataagcatattgacgcatac
aaaacattcccaccaacagagcctaaaaaggacaaaaagaagaaggctgatgaaactcaagccttaccgcagagacagaag
aaacagcaaactgtgactcttcttcctgctgcagatttggatgatttctccaaacaattgcaacaatccatgagcagtgctgactca
actcaggcctaatctagagggcccgtttaaacccgctgatcagcctcgactgtgccttctagttgccagccatctgttgtttgcccc
tcccccgtgccttccttgaccctggaaggtgccactcccactgtcctttcctaataaaatgaggaaattgcatcgcattgtctgagta
ggtgtcattctattctggggggtggggtggggcaggacagcaagggggaggattgggaagacaatagcaggcatgctgggg
atgcggtgggctctatggcttctgaggcggaaagaaccagctggggctctagggggtatccccacgcgccctgtagcggcgc
attaagcgcggcgggtgtggtggttacgcgcagcgtgaccgctacacttgccagcgccctagcgcccgctcctttcgctttcttc
ccttcctttctcgccacgttcgccggctttccccgtcaagctctaaatcgggggctccctttagggttccgatttagtgctttacggc
acctcgaccccaaaaaacttgattagggtgatggttcacgtagtgggccatcgccctgatagacggtttttcgccctttgacgttg
gagtccacgttctttaatagtggactcttgttccaaactggaacaacactcaaccctatctcggtctattcttttgatttataagggattt
tgccgatttcggcctattggttaaaaaatgagctgatttaacaaaaatttaacgcgaattaattctgtggaatgtgtgtcagttagggt
gtggaaagtccccaggctccccagcaggcagaagtatgcaaagcatgcatctcaattagtcagcaaccaggtgtggaaagtcc
ccaggctccccagcaggcagaagtatgcaaagcatgcatctcaattagtcagcaaccatagtcccgcccctaactccgcccatc
ccgcccctaactccgcccagttccgcccattctccgccccatggctgactaattttttttatttatgcagaggccgaggccgcctct
gcctctgagctattccagaagtagtgaggaggcttttttggaggcctaggcttttgcaaaaagctcccgggagcttgtatatccatt
ttcggatctgatcaagagacaggatgaggatcgtttcgcatgattgaacaagatggattgcacgcaggttctccggccgcttggg
tggagaggctattcggctatgactgggcacaacagacaatcggctgctctgatgccgccgtgttccggctgtcagcgcagggg
cgcccggttctttttgtcaagaccgacctgtccggtgccctgaatgaactgcaggacgaggcagcgcggctatcgtggctggcc
acgacgggcgttccttgcgcagctgtgctcgacgttgtcactgaagcgggaagggactggctgctattgggcgaagtgccggg
gcaggatctcctgtcatctcaccttgctcctgccgagaaagtatccatcatggctgatgcaatgcggcggctgcatacgcttgatc
cggctacctgcccattcgaccaccaagcgaaacatcgcatcgagcgagcacgtactcggatggaagccggtcttgtcgatcag
gatgatctggacgaagagcatcaggggctcgcgccagccgaactgttcgccaggctcaaggcgcgcatgcccgacggcga
ggatctcgtcgtgacccatggcgatgcctgcttgccgaatatcatggtggaaaatggccgcttttctggattcatcgactgtggcc
ggctgggtgtggcggaccgctatcaggacatagcgttggctacccgtgatattgctgaagagcttggcggcgaatgggctgac
cgcttcctcgtgctttacggtatcgccgctcccgattcgcagcgcatcgccttctatcgccttcttgacgagttcttctgagcggga
ctctggggttcgaaatgaccgaccaagcgacgcccaacctgccatcacgagatttcgattccaccgccgccttctatgaaaggtt
gggcttcggaatcgttttccgggacgccggctggatgatcctccagcgcggggatctcatgctggagttcttcgcccaccccaa
cttgtttattgcagcttataatggttacaaataaagcaatagcatcacaaatttcacaaataaagcatttttttcactgcattctagttgtg
gtttgtccaaactcatcaatgtatcttatcatgtctgtataccgtcgacctctagctagagcttggcgtaatcatggtcatagctgtttc
ctgtgtgaaattgttatccgctcacaattccacacaacatacgagccggaagcataaagtgtaaagcctggggtgcctaatgagt
gagctaactcacattaattgcgttgcgctcactgcccgctttccagtcgggaaacctgtcgtgccagctgcattaatgaatcggcc
aacgcgcggggagaggcggtttgcgtattgggcgctcttccgcttcctcgctcactgactcgctgcgctcggtcgttcggctgc
ggcgagcggtatcagctcactcaaaggcggtaatacggttatccacagaatcaggggataacgcaggaaagaacatgtgagc
aaaaggccagcaaaaggccaggaaccgtaaaaaggccgcgttgctggcgtttttccataggctccgcccccctgacgagcat
cacaaaaatcgacgctcaagtcagaggtggcgaaacccgacaggactataaagataccaggcgtttccccctggaagctccct
cgtgcgctctcctgttccgaccctgccgcttaccggatacctgtccgcctttctcccttcgggaagcgtggcgctttctcatagctc
acgctgtaggtatctcagttcggtgtaggtcgttcgctccaagctgggctgtgtgcacgaaccccccgttcagcccgaccgctgc
gccttatccggtaactatcgtcttgagtccaacccggtaagacacgacttatcgccactggcagcagccactggtaacaggatta
gcagagcgaggtatgtaggcggtgctacagagttcttgaagtggtggcctaactacggctacactagaagaacagtatttggtat
ctgcgctctgctgaagccagttaccttcggaaaaagagttggtagctcttgatccggcaaacaaaccaccgctggtagcggttttt
ttgtttgcaagcagcagattacgcgcagaaaaaaaggatctcaagaagatcctttgatcttttctacggggtctgacgctcagtgg
aacgaaaactcacgttaagggattttggtcatgagattatcaaaaaggatcttcacctagatccttttaaattaaaaatgaagttttaa
atcaatctaaagtatatatgagtaaacttggtctgacagttaccaatgcttaatcagtgaggcacctatctcagcgatctgtctatttc
gttcatccatagttgcctgactccccgtcgtgtagataactacgatacgggagggcttaccatctggccccagtgctgcaatgata
ccgcgagacccacgctcaccggctccagatttatcagcaataaaccagccagccggaagggccgagcgcagaagtggtcct
gcaactttatccgcctccatccagtctattaattgttgccgggaagctagagtaagtagttcgccagttaatagtttgcgcaacgttg
ttgccattgctacaggcatcgtggtgtcacgctcgtcgtttggtatggcttcattcagctccggttcccaacgatcaaggcgagtta
catgatcccccatgttgtgcaaaaaagcggttagctccttcggtcctccgatcgttgtcagaagtaagttggccgcagtgttatcac
tcatggttatggcagcactgcataattctcttactgtcatgccatccgtaagatgcttttctgtgactggtgagtactcaaccaagtca
ttctgagaatagtgtatgcggcgaccgagttgctcttgcccggcgtcaatacgggataataccgcgccacatagcagaactttaa
aagtgctcatcattggaaaacgttcttcggggcgaaaactctcaaggatcttaccgctgttgagatccagttcgatgtaacccactc
gtgcacccaactgatcttcagcatcttttactttcaccagcgtttctgggtgagcaaaaacaggaaggcaaaatgccgcaaaaaa
gggaataagggcgacacggaaatgttgaatactcatactcttcctttttcaatattattgaagcatttatcagggttattgtctcatgag
cggatacatatttgaatgtatttagaaaaataaacaaataggggttccgcgcacatttccccgaaaagtgccacctgacgtc.
>pc3.1-Flag-SARS_COV_2-S.
gacggatcgggagatctcccgatcccctatggtgcactctcagtacaatctgctctgatgccgcatagttaagcc
agtatctgctccctgcttgtgtgttggaggtcgctgagtagtgcgcgagcaaaatttaagctacaacaaggcaaggcttgaccga
caattgcatgaagaatctgcttagggttaggcgttttgcgctgcttcgcgatgtacgggccagatatacgcgttgacattgattattg
actagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccat
tgacgtcaatgggtggagtatttacggtaaactgcccacttggcagtacatcaagtgtatcatatgccaagtacgccccctattga
cgtcaatgacggtaaatggcccgcctggcattatgcccagtacatgaccttatgggactttcctacttggcagtacatctacgtatt
agtcatcgctattaccatggtgatgcggttttggcagtacatcaatgggcgtggatagcggtttgactcacggggatttccaagtct
ccaccccattgacgtcaatgggagtttgttttggcaccaaaatcaacgggactttccaaaatgtcgtaacaactccgccccattga
cgcaaatgggcggtaggcgtgtacggtgggaggtctatataagcagagctctctggctaactagagaacccactgcttactggc
ttatcgaaattaatacgactcactatagggagacccagctagcgtttaaacttaagcttggtaccgccatggactacaaggacgac
gacgacaaggggcggccgccctttgtttttcttgttttattgccactagtctctagtcagtgtgttaatcttacaaccagaactcaatta
ccccctgcatacactaattctttcacacgtggtgtttattaccctgacaaagttttcagatcctcagttttacattcaactcaggacttgt
tcttacctttcttttccaatgttacttggttccatgctatacatgtctctgggaccaatggtactaagaggtttgataaccctgtcctacc
atttaatgatggtgtttattttgcttccactgagaagtctaacataataagaggctggatttttggtactactttagattcgaagaccca
gtccctacttattgttaataacgctactaatgttgttattaaagtctgtgaatttcaattttgtaatgatccatttttgggtgtttattaccac
aaaaacaacaaaagttggatggaaagtgagttcagagtttattctagtgcgaataattgcacttttgaatatgtctctcagccttttctt
atggaccttgaaggaaaacagggtaatttcaaaaatcttagggaatttgtgtttaagaatattgatggttattttaaaatatattctaag
cacacgcctattaatttagtgcgtgatctccctcagggtttttcggctttagaaccattggtagatttgccaataggtattaacatcact
aggtttcaaactttacttgctttacatagaagttatttgactcctggtgattcttcttcaggttggacagctggtgctgcagcttattatgt
gggttatcttcaacctaggacttttctattaaaatataatgaaaatggaaccattacagatgctgtagactgtgcacttgaccctctct
cagaaacaaagtgtacgttgaaatccttcactgtagaaaaaggaatctatcaaacttctaactttagagtccaaccaacagaatcta
ttgttagatttcctaatattacaaacttgtgcccttttggtgaagtttttaacgccaccagatttgcatctgtttatgcttggaacaggaa
gagaatcagcaactgtgttgctgattattctgtcctatataattccgcatcattttccacttttaagtgttatggagtgtctcctactaaat
taaatgatctctgctttactaatgtctatgcagattcatttgtaattagaggtgatgaagtcagacaaatcgctccagggcaaactgg
aaagattgctgattataattataaattaccagatgattttacaggctgcgttatagcttggaattctaacaatcttgattctaaggttggt
ggtaattataattacctgtatagattgtttaggaagtctaatctcaaaccttttgagagagatatttcaactgaaatctatcaggccggt
agcacaccttgtaatggtgttgaaggttttaattgttactttcctttacaatcatatggtttccaacccactaatggtgttggttaccaac
catacagagtagtagtactttcttttgaacttctacatgcaccagcaactgtttgtggacctaaaaagtctactaatttggttaaaaac
aaatgtgtcaatttcaacttcaatggtttaacaggcacaggtgttcttactgagtctaacaaaaagtttctgcctttccaacaatttggc
agagacattgctgacactactgatgctgtccgtgatccacagacacttgagattcttgacattacaccatgttcttttggtggtgtca
gtgttataacaccaggaacaaatacttctaaccaggttgctgttctttatcaggatgttaactgcacagaagtccctgttgctattcat
gcagatcaacttactcctacttggcgtgtttattctacaggttctaatgtttttcaaacacgtgcaggctgtttaataggggctgaacat
gtcaacaactcatatgagtgtgacatacccattggtgcaggtatatgcgctagttatcagactcagactaattctcctcggcgggc
acgtagtgtagctagtcaatccatcattgcctacactatgtcacttggtgcagaaaattcagttgcttactctaataactctattgccat
acccacaaattttactattagtgttaccacagaaattctaccagtgtctatgaccaagacatcagtagattgtacaatgtacatttgtg
gtgattcaactgaatgcagcaatcttttgttgcaatatggcagtttttgtacacaattaaaccgtgctttaactggaatagctgttgaac
aagacaaaaacacccaagaagtttttgcacaagtcaaacaaatttacaaaacaccaccaattaaagattttggtggttttaatttttc
acaaatattaccagatccatcaaaaccaagcaagaggtcatttattgaagatctacttttcaacaaagtgacacttgcagatgctgg
cttcatcaaacaatatggtgattgccttggtgatattgctgctagagacctcatttgtgcacaaaagtttaacggccttactgttttgcc
acctttgctcacagatgaaatgattgctcaatacacttctgcactgttagcgggtacaatcacttctggttggacctttggtgcaggt
gctgcattacaaataccatttgctatgcaaatggcttataggtttaatggtattggagttacacagaatgttctctatgagaaccaaaa
attgattgccaaccaatttaatagtgctattggcaaaattcaagactcactttcttccacagcaagtgcacttggaaaacttcaagat
gtggtcaaccaaaatgcacaagctttaaacacgcttgttaaacaacttagctccaattttggtgcaatttcaagtgttttaaatgatat
cctttcacgtcttgacaaagttgaggctgaagtgcaaattgataggttgatcacaggcagacttcaaagtttgcagacatatgtgac
tcaacaattaattagagctgcagaaatcagagcttctgctaatcttgctgctactaaaatgtcagagtgtgtacttggacaatcaaaa
agagttgatttttgtggaaagggctatcatcttatgtccttccctcagtcagcacctcatggtgtagtcttcttgcatgtgacttatgtcc
ctgcacaagaaaagaacttcacaactgctcctgccatttgtcatgatggaaaagcacactttcctcgtgaaggtgtctttgtttcaaa
tggcacacactggtttgtaacacaaaggaatttttatgaaccacaaatcattactacagacaacacatttgtgtctggtaactgtgat
gttgtaataggaattgtcaacaacacagtttatgatcctttgcaacctgaattagactcattcaaggaggagttagataaatattttaa
gaatcatacatcaccagatgttgatttaggtgacatctctggcattaatgcttcagttgtaaacattcaaaaagaaattgaccgcctc
aatgaggttgccaagaatttaaatgaatctctcatcgatctccaagaacttggaaagtatgagcagtatataaaatggccatggtac
atttggctaggttttatagctggcttgattgccatagtaatggtgacaattatgctttgctgtatgaccagttgctgtagttgtctcaag
ggctgttgttcttgtggatcctgctgcaaatttgatgaagacgactctgagccagtgctcaaaggagtcaaattacattacacataa
gctctagagggcccgtttaaacccgctgatcagcctcgactgtgccttctagttgccagccatctgttgtttgcccctcccccgtgc
cttccttgaccctggaaggtgccactcccactgtcctttcctaataaaatgaggaaattgcatcgcattgtctgagtaggtgtcattct
attctggggggtggggtggggcaggacagcaagggggaggattgggaagacaatagcaggcatgctggggatgcggtggg
ctctatggcttctgaggcggaaagaaccagctggggctctagggggtatccccacgcgccctgtagcggcgcattaagcgcg
gcgggtgtggtggttacgcgcagcgtgaccgctacacttgccagcgccctagcgcccgctcctttcgctttcttcccttcctttctc
gccacgttcgccggctttccccgtcaagctctaaatcgggggctccctttagggttccgatttagtgctttacggcacctcgaccc
caaaaaacttgattagggtgatggttcacgtagtgggccatcgccctgatagacggtttttcgccctttgacgttggagtccacgtt
ctttaatagtggactcttgttccaaactggaacaacactcaaccctatctcggtctattcttttgatttataagggattttgccgatttcg
gcctattggttaaaaaatgagctgatttaacaaaaatttaacgcgaattaattctgtggaatgtgtgtcagttagggtgtggaaagtc
cccaggctccccagcaggcagaagtatgcaaagcatgcatctcaattagtcagcaaccaggtgtggaaagtccccaggctccc
cagcaggcagaagtatgcaaagcatgcatctcaattagtcagcaaccatagtcccgcccctaactccgcccatcccgcccctaa
ctccgcccagttccgcccattctccgccccatggctgactaattttttttatttatgcagaggccgaggccgcctctgcctctgagct
attccagaagtagtgaggaggcttttttggaggcctaggcttttgcaaaaagctcccgggagcttgtatatccattttcggatctgat
caagagacaggatgaggatcgtttcgcatgattgaacaagatggattgcacgcaggttctccggccgcttgggtggagaggct
attcggctatgactgggcacaacagacaatcggctgctctgatgccgccgtgttccggctgtcagcgcaggggcgcccggttct
ttttgtcaagaccgacctgtccggtgccctgaatgaactgcaggacgaggcagcgcggctatcgtggctggccacgacgggc
gttccttgcgcagctgtgctcgacgttgtcactgaagcgggaagggactggctgctattgggcgaagtgccggggcaggatct
cctgtcatctcaccttgctcctgccgagaaagtatccatcatggctgatgcaatgcggcggctgcatacgcttgatccggctacct
gcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacggcgaggatctcgtc
gtgacccatggcgatgcctgcttgccgaatatcatggtggaaaatggccgcttttctggattcatcgactgtggccggctgggtgt
ggcggaccgctatcaggacatagcgttggctacccgtgatattgctgaagagcttggcggcgaatgggctgaccgcttcctcgt
gctttacggtatcgccgctcccgattcgcagcgcatcgccttctatcgccttcttgacgagttcttctgagcgggactctggggttc
gaaatgaccgaccaagcgacgcccaacctgccatcacgagatttcgattccaccgccgccttctatgaaaggttgggcttcgga
atcgttttccgggacgccggctggatgatcctccagcgcggggatctcatgctggagttcttcgcccaccccaacttgtttattgc
agcttataatggttacaaataaagcaatagcatcacaaatttcacaaataaagcatttttttcactgcattctagttgtggtttgtccaa
actcatcaatgtatcttatcatgtctgtataccgtcgacctctagctagagcttggcgtaatcatggtcatagctgtttcctgtgtgaaa
ttgttatccgctcacaattccacacaacatacgagccggaagcataaagtgtaaagcctggggtgcctaatgagtgagctaactc
acattaattgcgttgcgctcactgcccgctttccagtcgggaaacctgtcgtgccagctgcattaatgaatcggccaacgcgcgg
ggagaggcggtttgcgtattgggcgctcttccgcttcctcgctcactgactcgctgcgctcggtcgttcggctgcggcgagcggt
atcagctcactcaaaggcggtaatacggttatccacagaatcaggggataacgcaggaaagaacatgtgagcaaaaggccag
caaaaggccaggaaccgtaaaaaggccgcgttgctggcgtttttccataggctccgcccccctgacgagcatcacaaaaatcg
acgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcatagctcacgctgtaggt
atctcagttcggtgtaggtcgttcgctccaagctgggctgtgtgcacgaaccccccgttcagcccgaccgctgcgccttatccgg
taactatcgtcttgagtccaacccggtaagacacgacttatcgccactggcagcagccactggtaacaggattagcagagcgag
gtatgtaggcggtgctacagagttcttgaagtggtggcctaactacggctacactagaagaacagtatttggtatctgcgctctgct
gaagccagttaccttcggaaaaagagttggtagctcttgatccggcaaacaaaccaccgctggtagcggtttttttgtttgcaagc
agcagattacgcgcagaaaaaaaggatctcaagaagatcctttgatcttttctacggggtctgacgctcagtggaacgaaaactc
acgttaagggattttggtcatgagattatcaaaaaggatcttcacctagatccttttaaattaaaaatgaagttttaaatcaatctaaag
tatatatgagtaaacttggtctgacagttaccaatgcttaatcagtgaggcacctatctcagcgatctgtctatttcgttcatccatagt
tgcctgactccccgtcgtgtagataactacgatacgggagggcttaccatctggccccagtgctgcaatgataccgcgagaccc
acgctcaccggctccagatttatcagcaataaaccagccagccggaagggccgagcgcagaagtggtcctgcaactttatccg
cctccatccagtctattaattgttgccgggaagctagagtaagtagttcgccagttaatagtttgcgcaacgttgttgccattgctac
aggcatcgtggtgtcacgctcgtcgtttggtatggcttcattcagctccggttcccaacgatcaaggcgagttacatgatcccccat
gttgtgcaaaaaagcggttagctccttcggtcctccgatcgttgtcagaagtaagttggccgcagtgttatcactcatggttatggc
agcactgcataattctcttactgtcatgccatccgtaagatgcttttctgtgactggtgagtactcaaccaagtcattctgagaatagt
gtatgcggcgaccgagttgctcttgcccggcgtcaatacgggataataccgcgccacatagcagaactttaaaagtgctcatcat
tggaaaacgttcttcggggcgaaaactctcaaggatcttaccgctgttgagatccagttcgatgtaacccactcgtgcacccaact
gatcttcagcatcttttactttcaccagcgtttctgggtgagcaaaaacaggaaggcaaaatgccgcaaaaaagggaataaggg
cgacacggaaatgttgaatactcatactcttcctttttcaatattattgaagcatttatcagggttattgtctcatgagcggatacatattt
gaatgtatttagaaaaataaacaaataggggttccgcgcacatttccccgaaaagtgccacctgacgtc.
>pcDNA3.1-Flag-SARS_COV_2_M(INfusion).
gacggatcgggagatctcccgatcccctatggtgcactctcagtacaatctgctctgatgccgcatagttaagcc
agtatctgctccctgcttgtgtgttggaggtcgctgagtagtgcgcgagcaaaatttaagctacaacaaggcaaggcttgaccga
caattgcatgaagaatctgcttagggttaggcgttttgcgctgcttcgcgatgtacgggccagatatacgcgttgacattgattattg
actagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccat
tgacgtcaatgggtggagtatttacggtaaactgcccacttggcagtacatcaagtgtatcatatgccaagtacgccccctattga
cgtcaatgacggtaaatggcccgcctggcattatgcccagtacatgaccttatgggactttcctacttggcagtacatctacgtatt
agtcatcgctattaccatggtgatgcggttttggcagtacatcaatgggcgtggatagcggtttgactcacggggatttccaagtct
ccaccccattgacgtcaatgggagtttgttttggcaccaaaatcaacgggactttccaaaatgtcgtaacaactccgccccattga
cgcaaatgggcggtaggcgtgtacggtgggaggtctatataagcagagctctctggctaactagagaacccactgcttactggc
ttatcgaaattaatacgactcactatagggagacccagctagcgtttaaacttaagcttggtaccgccatggactacaaggacgac
gacgacaaggggcggccgcaagcagattccaacggtactattaccgttgaagagcttaaaaagctccttgaacaatggaacct
agtaataggtttcctattccttacatggatttgtcttctacaatttgcctatgccaacaggaataggtttttgtatataattaagttaattttc
ctctggctgttatggccagtaactttagcttgttttgtgcttgctgctgtttacagaataaattggatcaccggtggaattgctatcgca
atggcttgtcttgtaggcttgatgtggctcagctacttcattgcttctttcagactgtttgcgcgtacgcgttccatgtggtcattcaatc
cagaaactaacattcttctcaacgtgccactccatggcactattctgaccagaccgcttctagaaagtgaactcgtaatcggagct
gtgatccttcgtggacatcttcgtattgctggacaccatctaggacgctgtgacatcaaggacctgcctaaagaaatcactgttgct
acatcacgaacgctttcttattacaaattgggagcttcgcagcgtgtagcaggtgactcaggttttgctgcatacagtcgctacagg
attggcaactataaattaaacacagaccattccagtagcagtgacaatattgctttgcttgtacagtaatctagagggcccgtttaaa
cccgctgatcagcctcgactgtgccttctagttgccagccatctgttgtttgcccctcccccgtgccttccttgaccctggaaggtg
ccactcccactgtcctttcctaataaaatgaggaaattgcatcgcattgtctgagtaggtgtcattctattctggggggtggggtgg
ggcaggacagcaagggggaggattgggaagacaatagcaggcatgctggggatgcggtgggctctatggcttctgaggcgg
aaagaaccagctggggctctagggggtatccccacgcgccctgtagcggcgcattaagcgcggcgggtgtggtggttacgcg
cagcgtgaccgctacacttgccagcgccctagcgcccgctcctttcgctttcttcccttcctttctcgccacgttcgccggctttccc
cgtcaagctctaaatcgggggctccctttagggttccgatttagtgctttacggcacctcgaccccaaaaaacttgattagggtgat
ggttcacgtagtgggccatcgccctgatagacggtttttcgccctttgacgttggagtccacgttctttaatagtggactcttgttcca
aactggaacaacactcaaccctatctcggtctattcttttgatttataagggattttgccgatttcggcctattggttaaaaaatgagct
gatttaacaaaaatttaacgcgaattaattctgtggaatgtgtgtcagttagggtgtggaaagtccccaggctccccagcaggcag
aagtatgcaaagcatgcatctcaattagtcagcaaccaggtgtggaaagtccccaggctccccagcaggcagaagtatgcaaa
gcatgcatctcaattagtcagcaaccatagtcccgcccctaactccgcccatcccgcccctaactccgcccagttccgcccattct
ccgccccatggctgactaattttttttatttatgcagaggccgaggccgcctctgcctctgagctattccagaagtagtgaggagg
cttttttggaggcctaggcttttgcaaaaagctcccgggagcttgtatatccattttcggatctgatcaagagacaggatgaggatc
gtttcgcatgattgaacaagatggattgcacgcaggttctccggccgcttgggtggagaggctattcggctatgactgggcacaa
cagacaatcggctgctctgatgccgccgtgttccggctgtcagcgcaggggcgcccggttctttttgtcaagaccgacctgtccg
gtgccctgaatgaactgcaggacgaggcagcgcggctatcgtggctggccacgacgggcgttccttgcgcagctgtgctcga
cgttgtcactgaagcgggaagggactggctgctattgggcgaagtgccggggcaggatctcctgtcatctcaccttgctcctgc
cgagaaagtatccatcatggctgatgcaatgcggcggctgcatacgcttgatccggctacctgcccattcgaccaccaagcgaa
acatcgcatcgagcgagcacgtactcggatggaagccggtcttgtcgatcaggatgatctggacgaagagcatcaggggctc
gcgccagccgaactgttcgccaggctcaaggcgcgcatgcccgacggcgaggatctcgtcgtgacccatggcgatgcctgct
tgccgaatatcatggtggaaaatggccgcttttctggattcatcgactgtggccggctgggtgtggcggaccgctatcaggacat
agcgttggctacccgtgatattgctgaagagcttggcggcgaatgggctgaccgcttcctcgtgctttacggtatcgccgctccc
gattcgcagcgcatcgccttctatcgccttcttgacgagttcttctgagcgggactctggggttcgaaatgaccgaccaagcgac
gcccaacctgccatcacgagatttcgattccaccgccgccttctatgaaaggttgggcttcggaatcgttttccgggacgccggc
tggatgatcctccagcgcggggatctcatgctggagttcttcgcccaccccaacttgtttattgcagcttataatggttacaaataaa
gcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccaca
caacatacgagccggaagcataaagtgtaaagcctggggtgcctaatgagtgagctaactcacattaattgcgttgcgctcactg
cccgctttccagtcgggaaacctgtcgtgccagctgcattaatgaatcggccaacgcgcggggagaggcggtttgcgtattgg
gcgctcttccgcttcctcgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggccaggaaccgtaaa
aaggccgcgttgctggcgtttttccataggctccgcccccctgacgagcatcacaaaaatcgacgctcaagtcagaggtggcg
aaacccgacaggactataaagataccaggcgtttccccctggaagctccctcgtgcgctctcctgttccgaccctgccgcttacc
ggatacctgtccgcctttctcccttcgggaagcgtggcgctttctcatagctcacgctgtaggtatctcagttcggtgtaggtcgttc
gctccaagctgggctgtgtgcacgaaccccccgttcagcccgaccgctgcgccttatccggtaactatcgtcttgagtccaaccc
ggtaagacacgacttatcgccactggcagcagccactggtaacaggattagcagagcgaggtatgtaggcggtgctacagagt
tcttgaagtggtggcctaactacggctacactagaagaacagtatttggtatctgcgctctgctgaagccagttaccttcggaaaa
agagttggtagctcttgatccggcaaacaaaccaccgctggtagcggtttttttgtttgcaagcagcagattacgcgcagaaaaa
aaggatctcaagaagatcctttgatcttttctacggggtctgacgctcagtggaacgaaaactcacgttaagggattttggtcatga
gattatcaaaaaggatcttcacctagatccttttaaattaaaaatgaagttttaaatcaatctaaagtatatatgagtaaacttggtctg
acagttaccaatgcttaatcagtgaggcacctatctcagcgatctgtctatttcgttcatccatagttgcctgactccccgtcgtgtag
ataactacgatacgggagggcttaccatctggccccagtgctgcaatgataccgcgagacccacgctcaccggctccagattta
tcagcaataaaccagccagccggaagggccgagcgcagaagtggtcctgcaactttatccgcctccatccagtctattaattgtt
gccgggaagctagagtaagtagttcgccagttaatagtttgcgcaacgttgttgccattgctacaggcatcgtggtgtcacgctcg
tcgtttggtatggcttcattcagctccggttcccaacgatcaaggcgagttacatgatcccccatgttgtgcaaaaaagcggttagc
tccttcggtcctccgatcgttgtcagaagtaagttggccgcagtgttatcactcatggttatggcagcactgcataattctcttactgt
catgccatccgtaagatgcttttctgtgactggtgagtactcaaccaagtcattctgagaatagtgtatgcggcgaccgagttgctc
ttgcccggcgtcaatacgggataataccgcgccacatagcagaactttaaaagtgctcatcattggaaaacgttcttcggggcga
aaactctcaaggatcttaccgctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacggaaatgttgaatactc
atactcttcctttttcaatattattgaagcatttatcagggttattgtctcatgagcggatacatatttgaatgtatttagaaaaataaaca
aataggggttccgcgcacatttccccgaaaagtgccacctgacgtc.

1. A cell-mediated sars-cov-2 vaccine, comprising: stem cells comprising vector containing the nucleic acids encoding S protein, M protein, and N protein of SARS-COV-2.

2-4. (canceled)

5. The cell-mediated sars-cov-2 vaccine of claim 1, wherein the stem cells are selected from the group consisting of human embryonic kidney cells and mesenchymal stem cells derived from human umbilical cord.

6. The cell-mediated sars-cov-2 vaccine of claim 1, wherein the vector is a eukaryotic expression vector.

7. The cell-mediated sars-cov-2 vaccine of claim 6, wherein the vector is a eukaryotic transient expression pc3.1 vector.

8. The cell-mediated sars-cov-2 vaccine of claim 1, wherein the vector comprises the S protein, M protein, and N protein together or the vector separately comprises the S protein, M protein, and N protein respectively.

9. A method for preparing a stem cell-mediated SARS-Cov-2 vaccine, comprising:
a) introducing nucleic acids encoding S protein, M protein, and N protein of SARS-COV-2 into a vector, and
b) introducing the vector into stem cells.

10. The method of claim 9, wherein the stem cells are selected from the group consisting of human embryonic kidney cells and mesenchymal stem cells derived from human umbilical cord.

11. The method of claim 9, wherein the vector is a eukaryotic expression vector.

12. The method of claim 11, wherein the vector is eukaryotic transient expression pc3.1 vector.

13. The method of claim 9, wherein introducing a nucleic acid encoding SARS-COV-2 antigenic protein of the SARS-COV-2 into a vector comprises introducing the nucleic acids encoding S protein, M protein, and N protein of SARS-COV-2 into a eukaryotic expression vector.

14. The method of claim 9, wherein the introducing the vector into stem cells comprises introducing a vector containing the nucleic acids encoding all S protein, M protein, and N protein or comprises introducing a vector containing nucleic acid encoding S protein, a vector containing nucleic acid encoding M protein and a vector containing nucleic acid encoding N protein.

15. The method of claim 9, wherein the introducing the vector into stem cells comprises transfecting the vector into the stem cells using liposome.

16. The method of claim 15, wherein the transfecting the vector into the stem cells using liposome is transfecting in a ratio of liposome volume to the amount of vector of 15 μL:3 μg.

17. The method of claim 9, further comprising recovering the stem cells and adding the stem cells to PBS buffer without calcium and magnesium ions.

18. A method for preventing from SARS-COV-2 infection in a subject, comprising immunizing the subject with the cell-mediated sars-cov-2 vaccine of claim 1.
